(19) United States
(12) Patent Application Publication
(10) Pub. No.: US 2010/0016725 A1
(43) Pub. Date: Jan. 21, 2010

(54) MULTI-BEAM TRANSMIT ISOLATION

(75) Inventor: Karl E. Thiele, Andover, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/519,902

(22) PCT Filed: Dec. 19, 2007
(86) PCT No.: PCT/IB07/55235

§ 371 Date: Jun. 18, 2009

Related U.S. Application Data
(60) Provisional application No. 60/871,014, filed on Dec. 20, 2006.

(51) Int. Cl. A61B 8/14

(52) U.S. Cl. 600/447

(57) ABSTRACT

A method for isolating ultrasound transmit beams and reducing cross-transmit beam interference in a multi-beam system involves transmitting a first ultrasound beam at a first and second positive angle and transmitting a second ultrasound beam at a first and second negative angle. The method further involves receiving a first, second, third, and fourth composite signals, where each of the composite signals includes a return signal and a reflected component. The method further includes applying a finite impulse response filter to the first and third composite signals and the second and fourth composite signals to obtain an average of the first and second composite signals and an average of the second and fourth composite signals and remove the reflected components.

The invention relates generally to ultrasound imaging using multiple ultrasound transmit beams, and more particularly to isolating ultrasound transmit beams and reducing cross-transmit beam interference in a multi-beam system using a Doppler method.

Diagnostic Ultrasound is one of the most versatile, least expensive, and widely used diagnostic imaging modalities in use today. With the advent of three-dimensional ultrasound and Doppler Tissue Imaging (DTI), much effort has been invested in increasing the frame rate in ultrasound imaging. One particular method involves receive multi-line beam processing where numerous ultrasound receive beams are calculated for each transmit beam or event.

A problem with this method is that to receive energy along a given scan line direction, ultrasound transmit energy needs to be supplied along that line of sight. To solve this problem, there are basically two approaches.

The first approach involves widening or “fattening” the transmit beam so that it encompasses a larger area or volume. This technique suffers from decreased resolution (both detailed and contrast) and from decreased sensitivity.

The second approach involves transmitting or “firing” multiple focused and compact transmit beams into the human body simultaneously. The problem with this method is cross-transmit beam interference (i.e., a form of cross-talk), that is, energy from one transmit beam contaminates the receive beams clustered along another transmit beam, and vice versa.

Several solutions have been presented to solve this problem of cross-transmit beam interference. Some of these solutions include aggressive nulling of the receive beamform to exclude energy from other transmit beams, coded excitation, spatial diversity, that is, placing the transmit beams as far apart as possible, and frequency diversity. For example, U.S. Pat. No. 6,179,780 describes various methods for overcoming the problem of cross talk, including using a receive beam synthesizer, using coded transmissions, using non-uniform scanning sequences, and using different transmit center frequencies. To the inventor's knowledge, these methods have not, as of yet, been employed commercially.

The present invention provides a solution to cross-transmit beam interference in a multi-beam system by providing a novel method of isolating the energy from the desired transmit beam, and the means for mitigating the energy and susceptibility to the “other” transmit beam(s).

The inventive method for isolating ultrasound transmit beams and reducing cross-transmit beam interference in a multi-beam system comprises the steps of performing a first transmit event by simultaneously transmitting at least two of ultrasound beams at disjoint spatial locations, each of the transmitted ultrasound beams generating an echo return; generating a sequence of transmit events; applying a phase factor to each of the transmitted ultrasound beams in each transmit event; in each successive transmit event, modulating the phase factor by a unique amount for each of the transmitted ultrasound beams; and, linearly combining the echo returns from two or more transmit events by constructively adding energy from a desired transmitted ultrasound beam and destructively interfering energy from the remaining transmitted ultrasound beams

The foregoing and other objects, aspects, features, and advantages of the invention will become more apparent from the following description and from the claims.

The invention is further described in the detailed description that follows, by reference to the noted drawings by way of non-limiting illustrative embodiments of the invention. As should be understood, however, the invention is not limited to the precise arrangements and instrumentalities shown. In the drawings, like reference characters generally refer to the same parts throughout the different views. Also, the drawings are not necessarily to scale, emphasis instead generally being placed upon illustrating the principles of the invention.

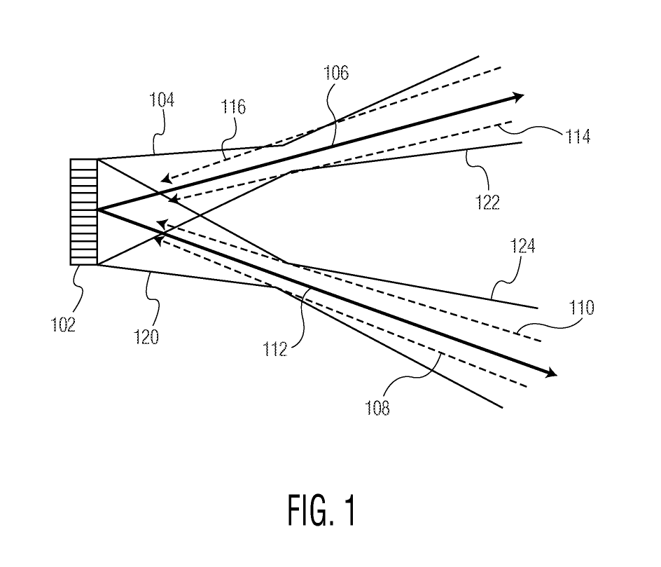

FIG. 1 is an illustrative schematic diagram of an ultrasound beam transmitter positioned to scan human tissue, according to one embodiment of the invention;

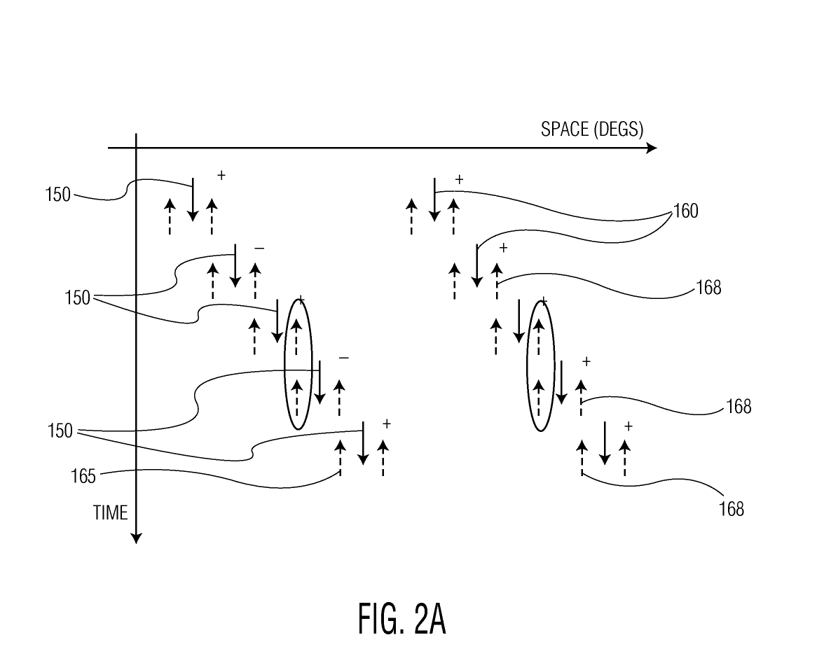

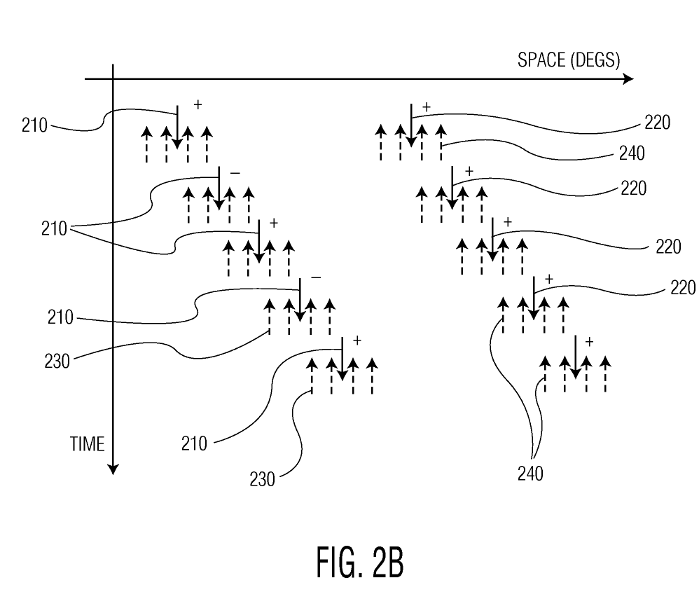

FIG. 2A is an illustrative schematic diagram of receive and transmit beams according to one embodiment of the invention;

FIG. 2B is an illustrative schematic diagram of receive and transmit beams, according to another embodiment of the invention;

FIG. 2C is an illustrative schematic diagram of receive and transmit beams according to another embodiment of the invention;

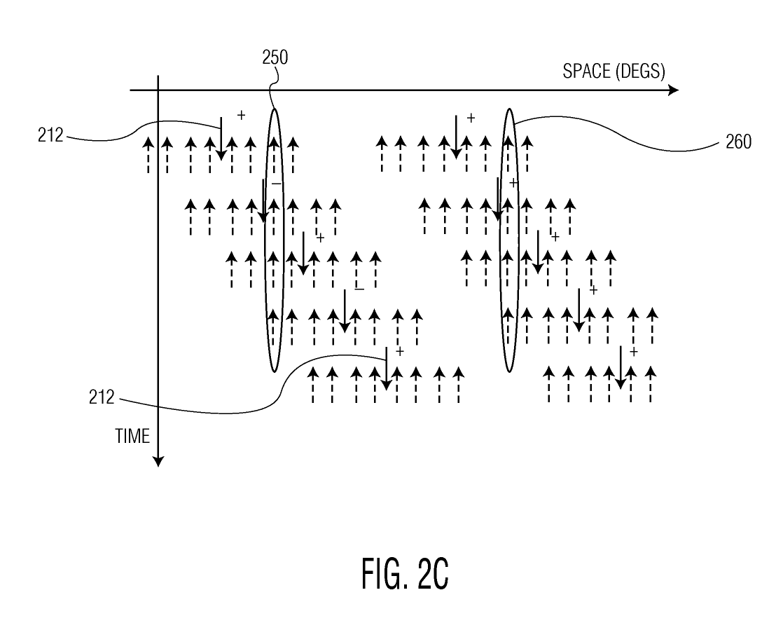

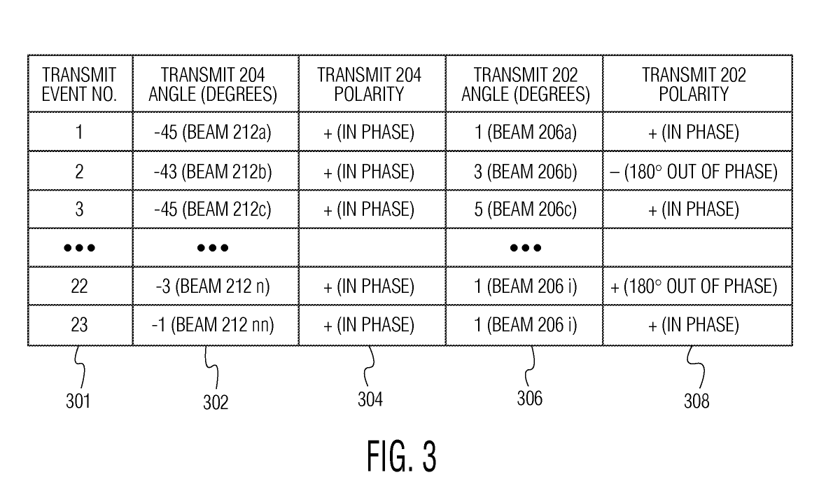

FIG. 3 is an illustrative table of ultrasound transmit events, angles, and polarities, according to one embodiment of the invention;

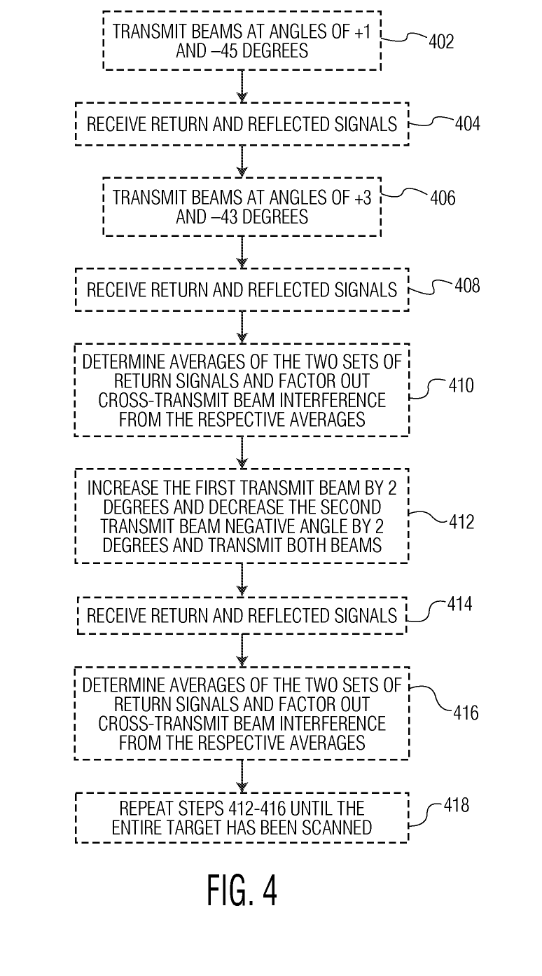

FIG. 4 is an illustrative flow diagram of a method for isolating transmit ultrasound beams and reducing cross-transmit beam interference in a multi-beam system, according to one embodiment of the invention;

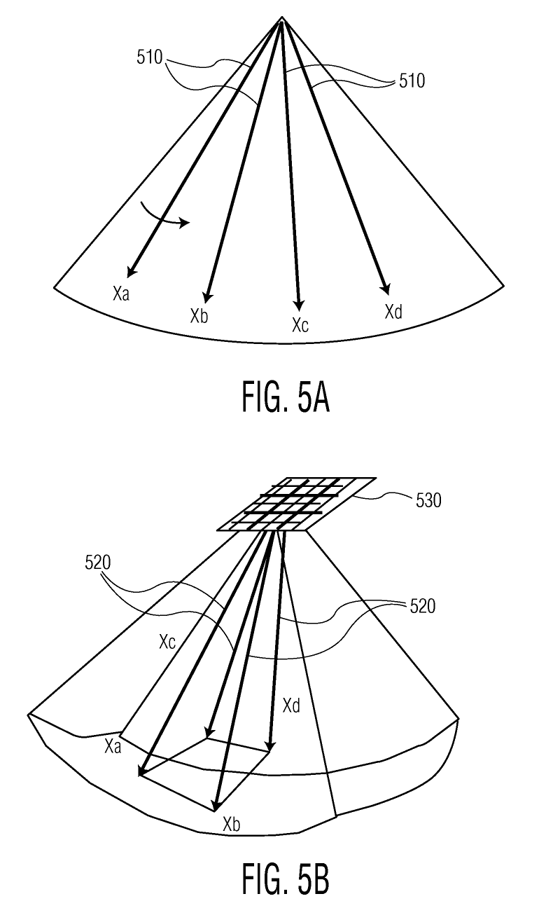

FIG. 5A is an illustrative schematic diagram of four simultaneous transmit beams which are co-planar for scanning a 2D image;

FIG. 5B is an illustrative schematic diagram of four simultaneous transmit beams which are non-planar for scanning volume;

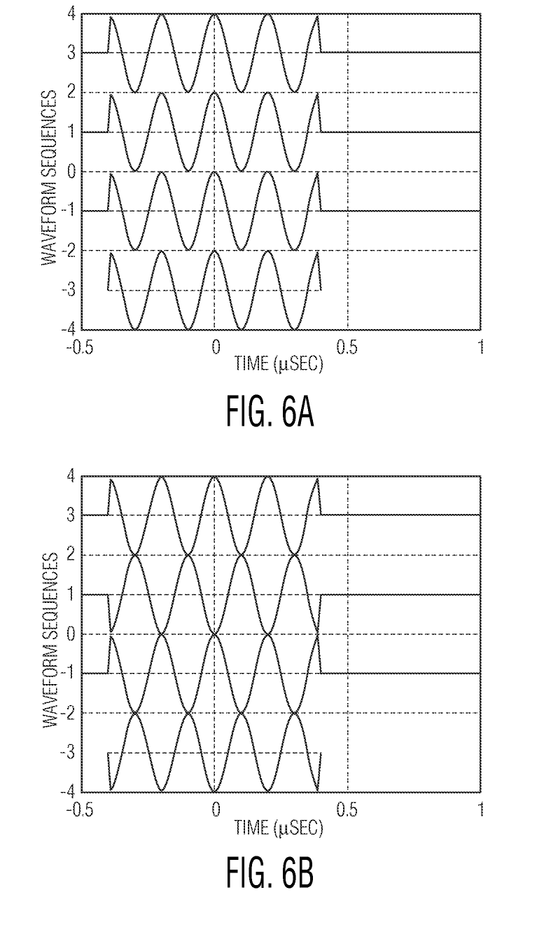

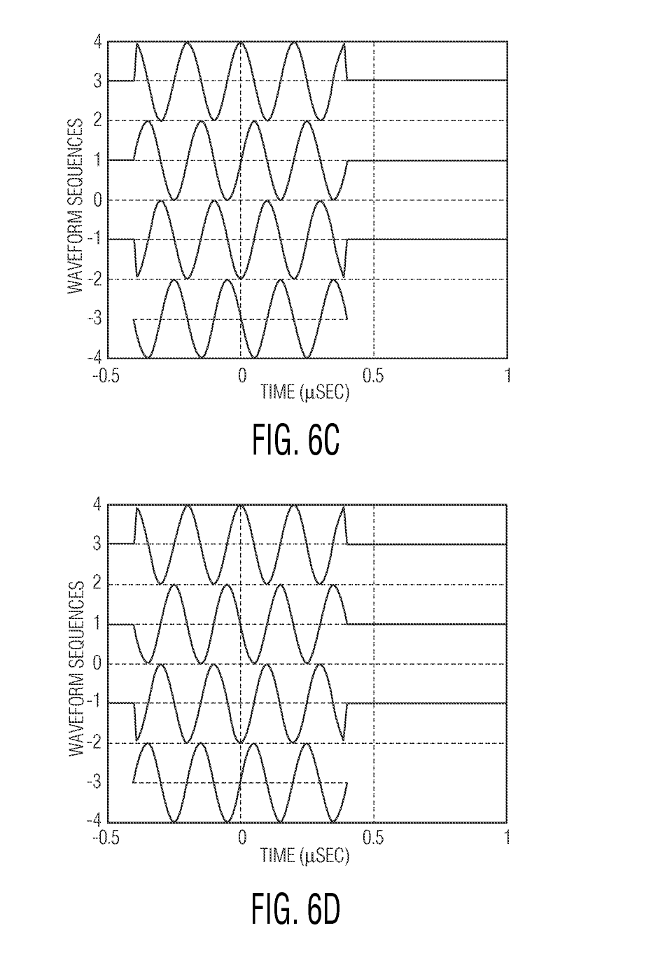

FIG. 6A shows transmit waveform sequences when the transmit waveforms are the same;

FIG. 6B shows transmit waveform sequences when the polarity toggles every other transmit;

FIG. 6C shows transmit waveform sequences when the transmit waveforms use an advancing phase term;

FIG. 6D shows transmit waveform sequences when the transmit waveforms use a retarding phase term;

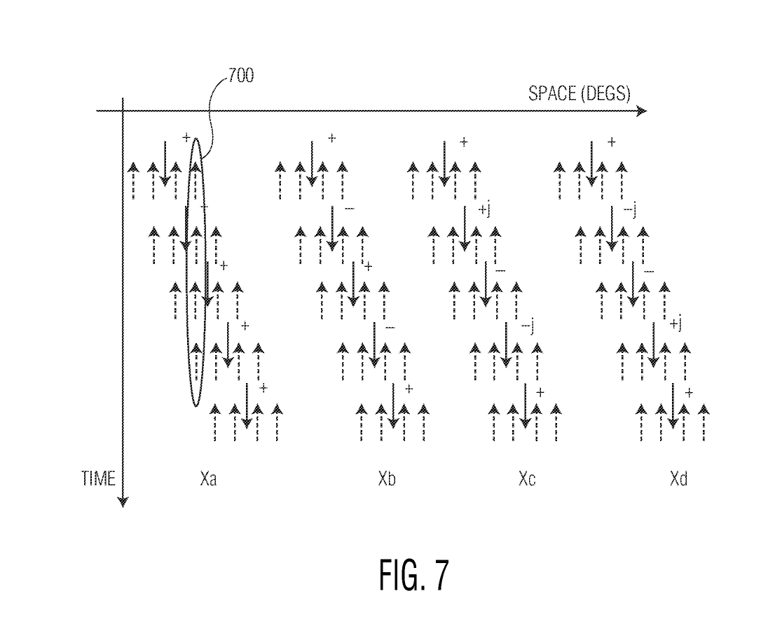

FIG. 7 is an illustrative schematic diagram of receive and transmit beams according to another embodiment of the invention;

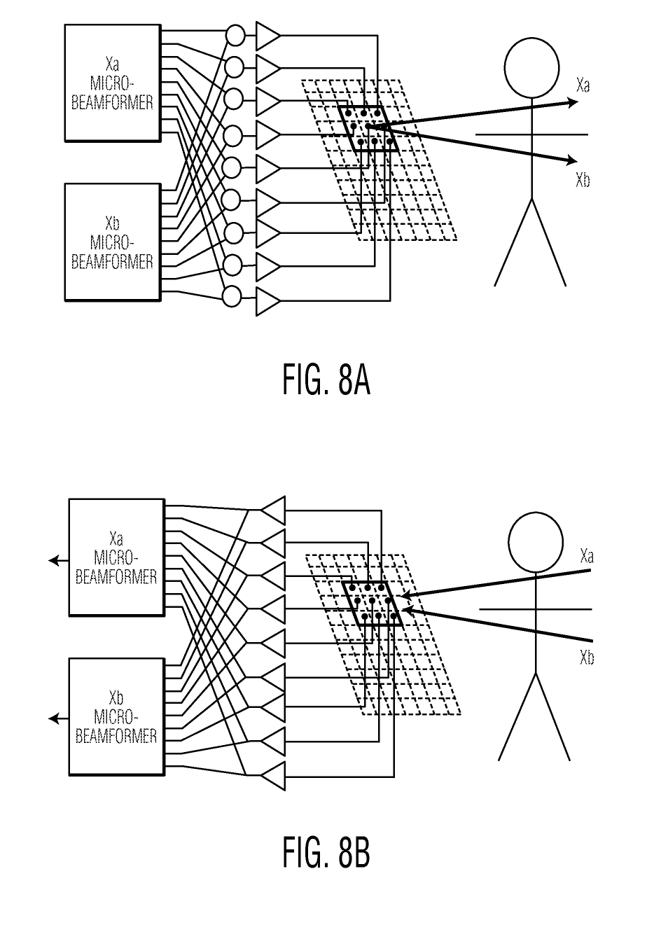

FIG. 8A is an illustrative schematic diagram of a distinct transmit wave field sending sound waves into a body; and

FIG. 8B is an illustrative schematic diagram of summing of patch echoes returning from a body.

Reference will now be made in detail to the preferred embodiments of the present invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be recognized by one of ordinary skill in the art that the present invention may be practiced without these specific details. In other instances, well known methods, procedures, components, and circuits have not been described in detail as not to unnecessarily obscure aspects of the present invention.

Referring to FIG. 1, in a simple embodiment, for each scan frame or scan volume, two simultaneous ultrasound transmit beams are employed; in other embodiments, more simultaneous ultrasound transmit beams are employed and will be discussed below. FIG. 1 shows an ultrasound transmitter/receiver 102 along with heavy solid arrows 106, 112 corresponding to the two simultaneous transmit beams which are positioned to scan human tissue. The solid lines 104, 120, 122, 124 surrounding these heavy lines with arrows 106, 112 illustrate the approximate 6 dB energy beamwidth, which effectively defines the width (resolution) of the transmit beam corresponding to that axial depth. Using dynamic receive beamforming, four simultaneous receive beams 108, 110, 114, 116, illustrated by the arrows using dotted lines, are acquired. FIG. 1 contains two receive beams 114, 116, 108, 110 for each transmit beam 106, 112. Multiple simultaneous transmit events are fired to scan over the entire 2D image, or, in the case of volumes, to scan over both the lateral and elevation dimensions of the volume. The ultrasound transmitter 102 produces one ultrasound beam 106 at a positive forty-five degree angle, and another ultrasound beam 112 at a negative forty-five degree angle.

Using dynamic receive beamforming, receive beams 108 and 114 are acquired or received by the ultrasound transmitter/receiver 102. However, the receiver 102 also receives a beam or signal 116, which is a reflected component of return beam or signal 114. The signal 116 contaminates the return beam or signal 108. Likewise, the receiver 102 also receives a beam or signal 110, which is a reflected component of return signal 108. The signal 110 contaminates the return signal 114. This cross-contamination of return signals 108 and 114 is referred to as cross-transmit beam interference, and degrades the contrast resolution of the ultrasound image.

In order to remove the contaminating signals 114, and 116 from return signals 108, and 110, respectively, a two coefficient finite image response (FIR) is applied to each of the return signals 108, 110, 114, 116, according to the equations A and B shown below.

((B3+N1)+(B4+(−N2)))/2=((B3+B4)/2)+((N1−N2)/2)=average of B3 and B4 Equation A:

((B1+N3)−(((−B2)+N4))/2=((B1−B2)/2)−((N3−N4)/2)=average of B1 and B2 Equation B:

Where B1, B2, B3, B4 are transmit beams and N1, N2, N3, N4 are nodes.

In a simple embodiment such as that shown in FIG. 1, we can assume two receive beams or signals per transmit beam, and we can further assume that as the transmit beam sequence appears across the field of view, the receive beams will overlap. The simple table below illustrates the simple embodiment sequence.

| Transmit | | Transmit A | | | | Transmit B | | |
|---|---|---|---|---|---|---|---|---|
| Event | Transmit A | Polarity | Receive X | Receive Y | Transmit B | Polarity | Receive U | Receive V |
| 1 | −44 degs | + | −45 degs | −43 degs | 0 degs | + | −1 degs | 1 degs |
| 2 | −42 | − | −43 | −41 | 2 | + | 3 | 1 |
| 3 | −40 | + | −41 | −39 | 4 | + | 5 | 3 |
| 4 | −38 | − | −39 | −37 | 6 | + | 7 | 5 |

FIG. 2A corresponds to this simple table, illustrating a simple embodiment of the present invention. FIG. 2A shows the solid downward arrows corresponding to the transmit beams 150, 160, and the dotted upward arrows corresponding to the receive beam locations 165, 168. It is assumed that transmit events on the left 150 are toggling in polarity, whereas transmit events on the right 160 maintain the same polarity.

Thus, in this simple embodiment, we will only have round trip reconstructed beams at odd degree values (as corresponding to the example table above). Focusing only on the constructive interference of the “Good” or non-contaminated energy produces the following equations:

RT₋₄₃=(+R₄₃X₋₄₄−−R₋₄₃X₋₄₂)/2

RT₋₄₁=(−R₋₄₁X₋₄₂−+R₋₄₁X₋₄₀)/2

RT₋₃₉=(+R₃₉X₋₄₀−−R₃₉X₋₃₈)/2

Where:
RT₋₄₃ is Roundtrip beam location at −43 degrees R₄₃X₋₄₄ is The receive beam @−43 degrees associated with the transmit beam @−44 degrees.
And, simultaneously solving for the round trip associated with Transmit Beams “B”:

RT₁=(+R₁X₀++R₁X₂)/2

RT₃=(+R₃X₂++R₃X₄)/2

RT₅=(+R₅X₄++R₅X₆)/2

Note that the desired energy associated with RT₋₄₃ has the transmit toggling in polarity every other transmit beam (+, −, +, −). Hence the “minus” sign in its equation. Conversely, the sign to coherently add the energy for RT₁ is associated with transmit beams that are always of the same polarity. Hence coherent summation requires the receive beams be “summed”.

The above equations are an oversimplification of what really happens, because the negative degree roundtrip beams, e.g. RT₋₄₃, are also susceptible to “Bad” or contaminated energy from the positive degree transmit events, and vice versa. The following equation includes the effect of the “Bad” energy.

RT₋₄₃={(+R₋₄₃X₋₄₄+_{BAD}R₋₄₃X₁)−(−R₋₄₃X₋₄₂+_{BAD}R₋₄₃X₃)}/2

Rearranging the terms in this equation yields:

RT₋₄₃={(+R₋₄₃X₋₄₄+_{BAD}R₋₄₃X₁)−(−R₋₄₃X₋₄₂+_{BAD}R₋₄₃X₃)}/2

The desired “Good” energy in the first half of the equation adds coherently, whereas the “Bad” energy from the 2^{nd} half of the equation is appropriately destructed. This is easy to see for the other “negative” degreed angles.

The technique illustrated with the above equations will also work for the positive degreed roundtrip angles as shown below.

RT₁={(+R₁X₀+_{BAD}R₁X₋₄₄)+(+R₁X₂−_{BAD}R₁X₋₄₂)}/2

Rearranging the terms in this equation yields:

RT₁={(+R₁X₀+R₁X₂)+(_{BAD}R₁X₋₄₄−_{BAD}R₁X₋₄₂)}/2

Again one can see that the bad energy from the opposite side transmit beams are appropriately canceled out.

In more advanced and preferred embodiments, there will be numerous receive beams for each transmit event, and in the simple positive/negative polarity case, the span of the receive beams will overlap each other by fifty percent. FIG. 2B shows four receive beams per transmit beam wherein the span of the receive beams overlap each other by fifty percent. In FIG. 2B, as in FIG. 2A, the solid downward arrows correspond to the transmit beams 210, 220, and the dotted up-arrows correspond to the receive beam locations 230, 240. As with the simple embodiment, it is assumed that transmit events on the left 210 are toggling in polarity, whereas the transmit events on the right 240 maintain the same polarity.

In the embodiment shown in FIG. 2B, the cross-beam rejection is diminished, because to “interpolate” to the correct round beam location requires the use of coefficients such as ¼, ¾, which results in the correct placement of the “Good” energy, but the “Bad” energy is only diminished by 6 dB (by ½).

In a preferred embodiment, we would have eight or more receive beams per transmit beam, and the overlap would be seventy-five percent or greater. This is illustrated in FIG. 2C. The circled regions 250, 260 illustrate how the roundtrip beam is reconstructed from the same angled receive beam corresponding to four different transmit events 212. Because the round trip beam will have four different coefficients associated with it, i.e. a four tap interpolation filter, the ability to suppress the “Bad” energy from the other transmit beams will be improved.

The equation defining how to combine the receive beams for group 250 is:

RT₂₅₀=a*X₁R₇−b*X₂R₅+c*X₃R₃−d*X₄R₁

There are some constraints on the coefficients that should improve the performance and achieve the desired results.

Constrain #1: The sum of the coefficients should equal one:

a+b+c+d=1

This causes the average energy in the multiple receive beams to have unity gain.

Constrain #2: The coefficients should interpolate to a location between the X2 and X3 transmit beams, and should be specifically located closer to X2 (as is graphically indicated in FIG. 2C). Describing this in equation form yields:

1*a+2*b+3*c+4*d=2.25

Note that the 1, 2, 3, 4 correspond to the spatial locations of the transmit beams X1, X2, X3, and X4, and the value 2.25 corresponds to the desired location of the interpolated output.

Constrain #3: The coefficients need to cancel out the energy from non-toggling polarity transmit beams from group 260 in FIG. 2C. This can be achieved by toggling the polarity of the coefficients, and making sure that they sum to zero:

a−b+c−d=0

One solution that meets the above constraints is:

a=0.025

b=0.60

c=0.475

d=−0.10

For the group of receive lines defined by 255 (to right of group 250), the coefficients can be swapped, yielding:

RT₂₅₅=d*X₁R₇−c*X₂R₅+b*X₃R₃−a*X₄R₁

Note that swapping the coefficients will modify Constraint #2, such that the resultant output beam will interpolate to “2.75” (still between X2 and X3, but now closer to X3).

Likewise, these coefficients can be applied to the groups 260 and 265 (to right of 260):

RT₂₆₀=a*X₁₀₁R₇+b*X₁₀₂R₅+c*X₁₀₃R₃+d*X₁₀₄R₁

RT₂₆₅=d*X₁₀₁R₇+c*X₁₀₂R₅+b*X₁₀₃R₃+a*X₁₀₄R₁

Note the difference in the sign of the coefficients.

As will be obvious to one skilled in the art, the round trip beams defined by RT250, RT255, RT260, and RT265 will be accurately located and will reject leakage energy from the “other” group of transmit beams.

A further embodiment of this invention is its use in conjunction with U.S. Provisional Patent Application No. 60/747,148, titled “ULTRASONIC SYNTHETIC TRANSMIT FOCUSING WITH A MULTILINE BEAMFORMER”, incorporated herein by reference. In this case, one can describe the RT260 round-trip beam as follows:

RT₂₆₀(t)=a*X₁R₇(t−d₁)+b*X₂R₅(t−d₂)+c*X₃R₃(t−d₃)+d*X₄R₁(t−d₄)

In this equation, “t” refers to the time during which the ultrasound echoes are coming from increasing depths in the body, and the delays, d1, d2, d3, d4, are calculated to retrospectively beamform the transmit beam as defined in the above provisional patent application. By applying Constrain #3 (a−b+c−d=0) to the above RT₂₆₀(t) equation, one can achieve the benefits of improved transmit focusing and mitigation of energy from the undesired transmit beams.

Referring to FIG. 3, in one embodiment, a table of ultrasound transmit events 301 (instance of a transmitted signal) including angles of transmission 302, 304, and polarities 306, 308 of the transmitted signals is shown. For transmitter 204, the angles of transmission 302 increment from −45 degrees to −1 degrees in +2 degree increments, with the polarity 304 of the transmitted single remaining positive (i.e., in phase). For transmitter 202, the angles of transmission 306 increment from +1 degrees to +45 degrees in 2 degree increments, with the polarity 308 of the transmitted single switching from positive to negative (i.e., 180 degrees out of phase), such that every other signal transmission is 180 degrees out of phase with the previous signal transmission.

Referring to FIGS. 3 and 4, the previously described method is repeated for each pair of consecutive transmit beams for each transmitter 202 and 204. For example, transmitter/receiver 202 transmits beam 206a at a positive one-degree angle and transmitter 204 simultaneously transmits beam 212a at a negative forty-five degree angle (Step 402). Receiver 220 receives return signal 208a and reflected signal 216a and receiver 222 receives return signal 214a and reflected signal 210a (Step 404). Transmitter 202 next transmits beam 206b at a positive three-degree angle and transmitter 204 simultaneously transmits beam 212b at a negative forty-three degree angle (Step 406). Receiver 220 receives return signal 208b and reflected signal 216b and receiver 222 receives return signal 214b and reflected signal 210b (Step 408). A data processing unit, such as a computer, executes the signal averaging algorithm to determine the average of return signals 208a and 208b, and return signals 214a and 214b (Step 410).

Next, the transmitter 202 transmits a third beam at a positive five-degree angle and transmitter 204 transmits a simultaneous third beam at a negative forty-one degree angle (Step 412). Receiver 220 receives a third return signal and a third reflected signal, and receiver 222 also receives a third return signal and a third reflected signal (Step 414). The data processing unit again executes the signal averaging algorithm to determine the average of return signal 208b and the third return signal, and the average of the return signal 214b and the other third return signal (Step 412). This sequence of steps repeats until the desired tissue area (not shown) has been scanned.

The aforementioned embodiments all involve two simultaneous transmit beams, such that one sequence of beams maintains a normal polarity, while the second set of transmit beams toggle in polarity. An aspect of this invention is to support more than two transmit beams, such that for any given transmit beam sequence, energy from all other transmits is mitigated. The following example will demonstrate a simultaneous four beam sequence. Four simultaneous transmit beams 510 can be co-planar for scanning a 2D image, as is illustrated in FIG. 5A, or they can be non-planar 520, for scanning a volume, as is illustrated in FIG. 5B. To transmit non-planar transmit beams, a 2D Matrix transducer of elements 530 is used, as is shown in FIG. 5B. Note that the following example applies to both planar and non-planar cases. The rejection of “bad” energy occurs in the time domain, so it does not matter where in space the cross-contaminating transmit beam is located.

In one embodiment, assume there are four beam sequences, referred to as Xa, Xb, Xc, and Xd. Each beam will cover different portions of the scanned regions. Furthermore, each beam will proceed through four different transmit waveforms.

For Xa, shown in FIG. 6A, the transmit waveforms will be the same. These can be expressed as:

Xa(t,n=1)=cos(2*pi*f*t)*w(t)

Xa(t,n=2)=cos(2*pi*f*t)*w(t)

Xa(t,n=3)=cos(2*pi*f*t)*w(t)

Xa(t,n=4)=cos(2*pi*f*t)*w(t)

Note that “t” refers to time, “n” refers to the transmit event, “f” refers to the nominal transmit frequency (e.g. 5.0 MHz), and “w(t)” refers to a time windowing function. For the example in FIG. 6a, 6b, 6c, and 6d, w(t) can be a rectangular windowing function which is only on (=1) from −0.4 to +0.4 usec. At 5 MHz, this would result in a transmit waveform having only four cycles. It is assumed that w(t) is the same for all transmit sequences (Xa, Xb, Xc, and Xd). Furthermore, it is assumed that this four waveform sequence will repeat, such that the fifth waveform will use waveform #1: Xa(t,n=5)=Xa(t,n=1).

Also, for Xb, shown in FIG. 6B, the transmit waveforms will use the previous method where the polarity toggles every other transmit. This can be expressed as:

Xb(t,n=1)=+cos(2*pi*f*t)*w(t)

Xb(t,n=2)=−cos(2*pi*f*t)*w(t)

Xb(t,n=3)=+cos(2*pi*f*t)*w(t)

Xb(t,n=4)=−cos(2*pi*f*t)*w(t)

However, for Xc (and Xd), one needs yet another sequence that can be uniquely distinguished. In this case, one can advance (or retard) the “phase” of the transmit waveform. Xc, which uses an advancing phase term, as shown in FIG. 6C, can be expressed as:

Xc(t,n=1)=+cos(2*pi*f*t)*w(t)

Xc(t,n=2)=+sin(2*pi*f*t)*w(t)

Xc(t,n=3)=−cos(2*pi*f*t)*w(t)

Xc(t,n=4)=−sin(2*pi*f*t)*w(t)

And for Xd, which uses a retarding phase term, as seen in FIG. 6D, the expressions are:

Xd(t,n=1)=+cos(2*pi*f*t)*w(t)

Xd(t,n=2)=−sin(2*pi*f*t)*w(t)

Xd(t,n=3)=−cos(2*pi*f*t)*w(t)

Xd(t,n=4)=+sin(2*pi*f*t)*w(t)

For purposes of illustrating this particular embodiment, each of the four transmit beam sequences will simultaneously receive four beams per transmit, as is shown in FIG. 7. The following equation corresponds to the encircled group 700 of receive lines for transmit Xa:

RT_{XA@2.5}=a*X_{A1}R₄+b*X_{A2}R₃+c*X_{A3}R₂+d*X_{A4}R₁

Since there are more concurrent transmit beams than in prior embodiments, there will be some additional constraints on the selection of the a,b,c,d coefficients.

| | | |
|---|---|---|
| Constraint 1: | a + b + c + d = 1 | Sum Coherent Energy from Xa |
| Constraint 2: | a − b + c − d = 0 | Reject energy from Xb |
| Constraint 3: | a + jb − c − jd = 0 | Reject energy from Xc |
| Constraint 4: | a − jb − c + jd = 0 | Reject energy from Xd |

Note that “j” refers to the imaginary sqrt(−1), and corresponds to a 90 degree phase shift associated with transmits Xc and Xd.

Solving for a,b,c,d yields the very simple result:

a=b=c=d=0.25

For someone skilled in the art, it would be a simple matter to come up with similar sets of coefficients for the other transmits: Xb, Xc, and Xd.

FIG. 5B shows the use of a 2D Matrix transducer 530 to scan a volume using four simultaneous transmit beams. On matrix transducers, it is desired to use a fully sampled aperture (all of the elements electrically active) for improved image quality and sensitivity. This is compared to sparse arrays, which only connect a small percentage of the elements. Fully sampled arrays can be achieved by using micro-beamformers located in the housing of the matrix transducer. See U.S. Pat. Nos. 5,997,479 and 6,126,602 which are incorporated herein by reference. Each micro-beamformer will appropriately beamform a small subset of elements, referred to as a patch. As currently known to those skilled in the art, the use of micro-beamformers will be incompatible with simultaneous transmit beams, and with this invention. This is because each patch or group of elements is limited to a singular steer angle on both transmit and receive. And, implicit in this invention is the use of multiple transmits which can be spatially separated and non co-located.

Hence it is a further inventive aspect of this invention to allow simultaneous transmit beams to be used with matrix transducers using micro-beamformers. One inventive element replicates the micro-beamformer electronics, one for each simultaneous transmit beam. For example, in the case where two beams are simultaneously transmitted, there will be two micro-beamformers per patch (per group of elements). Each micro-beamformer will produce a distinct transmit wave field, will be combined with the transmit wave fields from the other micro-beamformers associated with a single patch, will be amplified, and will drive the patch elements to send sound waves into the body (see FIG. 8A). Additionally, on receive, the shared patch elements will convert the returning sound waves to electrical signals, will be amplified, and will be sent to the N distinct micro-beamformers. Each beamformer will then delay and sum the returning patch echoes in the direction associated with the direction used during transmit (see FIG. 8b). In the general case, there will need to be “N” micro-beamformers for “N” simultaneous transmit beam look directions.

It is implicit in all of the aforementioned embodiments that they were designed for use in a “fundamental” mode of black-and-white gray scale imaging. Fundamental mode is where the transmit frequency is the same as the receive frequency. There is another mode of operation, referred to as Tissue Harmonic Imaging (THI), which is quite common in the current clinical practice of diagnostic ultrasound. In THI, harmonic frequencies are generated during the transmission and propagation of the transmit waveforms. These harmonics (often the second harmonic) are then selectively isolated on receive using bandpass filters. For example, the transmit waveform might be centered at 2.5 MHz, and the receive filters are set to 5.0 MHz to selectively receive the desired second harmonic.

In THI, in order to reject cross-beam contamination from simultaneous transmit as described by this invention, one needs to control the transmit in such a way that the desired phase relationship is observed on receive. For example, in the 2× multi-beam transmit embodiment, it is desired that the first sequence of beams have common receive phase, whereas the second set of transmits have the polarity of the receive signal toggle by 180 degrees every other transmit. In order to achieve this 180 degree toggling on the receive harmonic, the transmits for this sequence need to toggle between 0 and 90 degrees. In other words, the transmit sequence would toggle between a windowed cosine burst and a windowed sine burst. In the 4× multi-beam transmit embodiment, the various transmit sequences would need to be advanced (or retarded) by 45 degrees to achieve the desired 90 degree shift on receive (for the second harmonic).

As would be known to someone skilled in the art, the transmit phase shift would be approximately 1/H of the desired phase shift observed on receive, where “H” refers to the receive harmonic. Also known to one skilled in the art is that this phase relationship is not always exact, and may need to be finely adjusted based upon empirical measurements.

In a preferred embodiment, the data processing unit can be an FPGA (field programmable gate array), or an ASIC (application specific integrated circuit). The processing can also be performed using DSPs (digital signal processing units) or other computational units. In a preferred embodiment, two transmitter/receivers are used with one of the transmission beams switching between zero and 180 degree phases. In other embodiments, three or more ultrasound transmitters are used with the transmission beams transmitting at 0, 90, 180, and 270 degree phases. In still another embodiment, one beam would always be in phase (zero degrees), one beam would advance at +90 increments, one beam would advance at −90 increments, and one beam would toggle between 0 and 180 degrees.

Variations, modifications, and other implementations of what is described herein may occur to those of ordinary skill in the art without departing from the spirit and scope of the invention. Accordingly, the invention is not to be defined only by the preceding illustrative description.

What is claimed is:

1. A method for isolating ultrasound transmit beams and reducing cross-transmit beam interference in a multi-beam system, the method comprising:
performing a first transmit event by simultaneously transmitting a number of ultrasound beams at disjoint spatial locations, said number being at least two, each of said at least two transmitted ultrasound beams generating an echo return;
generating a sequence of transmit events over time;
applying a phase factor to each of the at least two transmitted ultrasound beams in each transmit event; and
in each successive transmit event, modulating the phase factor by a unique amount for each transmitted ultrasound beams, wherein the echo returns from two or more transmit events are combined by constructively adding energy from a desired transmitted ultrasound beam and destructively interfering energy from the remaining transmitted ultrasound beams.

2. The method according to claim 1, wherein the number of transmitted ultrasound beams equals two transmitted ultrasound beams per transmit event, and wherein the phase factor simplifies to {+1+1+1 . . . } for one of the transmitted ultrasound beams, and simplifies to {+1−1+1−1 . . . } for the other transmitted ultrasound beams.

3. The method according to claim 1, wherein the disjoint spatial locations are defined in degrees associated with one of a phased array sector and a Curved Linear Array transducer.

4. The method according to claim 1, wherein the disjoint spatial locations are offset in lateral distances associated with a linear transducer.

5. The method according to claim 1, wherein the disjoint spatial locations correspond to different transmit focal depths.

6. The method according to claim 1, where the successive transmit events sequentially scan one of a 2D image and a 3D volume.

7. The method according to claim 1, wherein the at least two transmitted ultrasound beams are isolated after receive beamforming at a summing node.

8. The method according to claim 7, further using parallel processing during the receive beamforming for creating one or more receive beams for each of the at least two transmitted ultrasound beams.

9. The method according to claim 8, wherein each of the receive beams has a unique set of coefficients used to combine the energy from successive transmit events, wherein the energy from the desired transmitted ultrasound beam is constructively added, and the energy from the other undesired transmitted ultrasound beams are destructively interfered.

10. The method according to claim 1, wherein multi-beam system contains an ultrasound transducer utilizing micro-Beamforming electronics.

11. The method according to claim 10, wherein the micro-beamforming electronics beamforms at least one patch, and an intra-group processor for each patch is replicated N times for each of the N spatially disjoint transmitted ultrasound beams.

12. The method according to claim 1, wherein the phase factor modulations can be approximated using time delays on at least one of transmission and reception.

13. The method according to claim 1, wherein said phase factor is modulated using Tissue Harmonic Imaging.

14. The method according to claim 13, wherein said Tissue Harmonic Imaging comprises at least two harmonic components, and a phase factor modulation amount applied to the transmit beams is essentially halved, wherein an observed phase factor at 2×RF during reception is effectively doubled through a non-linear wave propagation associated with the second of the at least two harmonics.

15. The method according to claim 13, wherein for an Mth harmonic component of the transmitted waveform, a phase factor modulation amount applied to the transmit beams is essentially halved, wherein an observed phase factor observed at the Mth receive harmonic M×F×mit during reception is effectively doubled through a non-linear wave propagation associated with a second harmonic of the Tissue Harmonic Imaging.

16. A method for allowing faster frame rates in ultrasound imaging, the method comprising:
simultaneously transmitting multiple ultrasound beams using a matrix array ultrasound transducer having one or more micro-beamformers, wherein the matrix transducer comprises a 2D array of ultrasonic elements containing electronics in the transducer housing to perform some aspect of beamforming, the electronics in the transducer housing supporting independent and separate simultaneously transmitted ultrasound beams being beamformed in disjoint spatial locations.

17. The method according to claim 16, further comprising:
generating a sequence of transmit events over time, each transmit event comprising simultaneously transmitting a number of ultrasound beams at disjoint spatial locations, each of said transmitted ultrasound beams generating an echo return;
applying a phase factor to each of the transmitted ultrasound beams in each transmit event; and
in each successive transmit event, modulating the phase factor by a unique amount for each transmitted ultrasound beam, wherein the echo returns from two or more transmit events are combined by constructively adding energy from a desired transmitted ultrasound beam and destructively interfering energy from the remaining transmitted ultrasound beams.

18. The method according to claim 16, wherein at least two micro-beamformers having simultaneously transmitted beams comprise a patch.

19. The method according to claim 16, wherein each micro-beamformer produces a distinct transmit wave field, and the distinct transmit wave fields of the micro-beamformers in the patch are combinable.

20. The method according to claim 16, wherein an intra-group processor for each patch is replicated N times for each of the spatially disjointed transmitted ultrasound beams.

21. The method according to claim 16, wherein the phase factor modulations can be approximated using time delays on at least one of transmission and reception.
